(19) United States
(12) Patent Application Publication
(10) Pub. No.: US 2026/0027239 A1
(43) Pub. Date: Jan. 29, 2026

(54) cRGD -CONJUGATED IMAGING AGENT

(71) Applicant: Curadel Surgical Innovations, Inc., Natick, MA (US)

(72) Inventor: John V. Frangioni, Weston, MA (US)

(73) Assignee: Curadel Surgical Innovations, Inc., Natick, MA (US)

(21) Appl. No.: 19/143,603

(22) PCT Filed: Dec. 28, 2023
(86) PCT No.: PCT/US2023/086257

§ 371 Date: Jun. 26, 2025

Related U.S. Application Data
(60) Provisional application No. 63/436,170, filed on Dec. 30, 2022.

(51) Int. Cl. A61K 49/00

(52) U.S. Cl. CPC A61K 49/0056; A61K 49/0032; A61K 49/0052

(57) ABSTRACT

The present invention relates to an imaging agent comprising a ZW700-1c dye conjugated to a cyclic-RGD peptide targeting ligand. The conjugate of the invention has superior stability and desirable in vivo properties as compared to other zwitterionic near-infrared contrast agents. The invention also relates to a method of imaging cells of a subject using the imaging agent. In particular, the invention relates to a method of imaging or identifying cells of a subject which are or are suspected of overexpressing one or more integrins using the imaging agent. Such cells include tumors, inflammatory cells, and cells undergoing angiogenesis.

### CROSS REFERENCE TO RELATED APPLICATION

The present application claims priority to U.S. Provisional Patent Application Ser. No. 63/436,170, filed Dec. 30, 2022. The disclosure of the above-identified prior U.S. Patent Application, in its entirety, is considered as being part of the present application, and thus, is incorporated herein by reference in its entirety.

### FIELD OF THE INVENTION

The present invention relates to methods of optically imaging tissues or cells using imaging agents having desirable in vivo properties that result in improved signal-to-background ratio with increased stability as compared to other zwitterionic near-infrared contrast agents.

### BACKGROUND OF THE INVENTION

Near infrared (NIR) fluorescence has potential importance in the medical field, particularly in diagnostics and image-guided surgery. However, the availability of suitable fluorophores as imaging agents has been a primary hindrance. To be clinically viable, the ideal NIR fluorophore should have both good optical properties and superior in vivo properties with respect to solubility, biodistribution, and clearance. Known fluorophores tend to clear through the liver, which results in undesirable fluorescence throughout the gastrointestinal tract. And in some cases, known fluorophores suffer from significant nonspecific background uptake in normal tissues, resulting in a low signal-to-background ratio.

In recent years, more advanced fluorophores have been developed which result in improved signal-to-background. See, for example, U.S. Pat. Nos. 11,077,210, 9,687,567, 10,493,169, 10,201,621, and 10,478,512. Although these advanced fluorophores lower non-specific uptake in non-target tissue, they are relatively labile in blood, becoming metabolized in minutes to hours. As such, there remains a need for new and improved NIR fluorescent imaging agents with increased stability that can equilibrate rapidly between the intravascular and extravascular spaces and are cleared efficiently, including by renal filtration. The imaging agents of the invention are directed toward these and other needs.

### SUMMARY OF THE INVENTION

The invention is based, at least in part, on the discovery that replacement of a key bond in the near-infrared fluorophore with a carbon-carbon bond conjugation to a cyclic cRGD (cRGD) peptide as a targeting ligand results in vastly increased stability as compared to NIR fluorophores without such a bond, while preserving ligand and zwitterionic properties.

One of the most stable fluorophores currently in use is known as ZW700-1c. This is also referred to as ZW700-1-Forte.

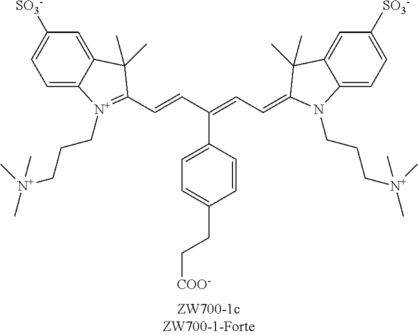

This is a 700-nm zwitterionic pentamethine indocyanine near-infrared fluorophore which permits dual-channel image-guided surgery.

In one aspect, the present invention provides methods of imaging tissue or cells, the methods including (a) contacting the tissue or cells with an imaging agent comprising a conjugate of ZW700-1c and a cRDG peptide; (b) irradiating the tissue or cells at a wavelength absorbed by the conjugate; (c) detecting an optical signal from the irradiated tissue or cells, wherein the signal-to-background ratio of the detected optical signal is at least about 1.1, thereby imaging the tissue or cells.

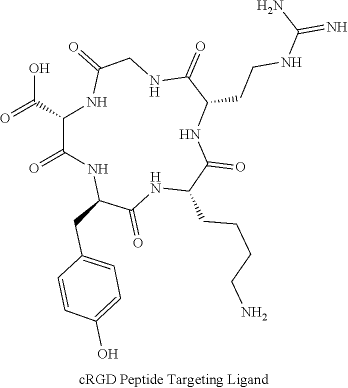 [SMILES: N=C(N)NCC[C@@H]1NC(=O)[C@H](CCCCN)NC(=O)[C@@H](Cc2ccc(O)cc2)NC(=O)[C@H](C(=O)O)NC(=O)CNC1=O]

The imaging agent of the invention is particularly advantageous because their behavior in vivo is believed to contribute to superior optical imaging properties and superior stability. More specifically, the charge-balancing is believed to impart good biodistribution and clearance properties to the agents, and reduce undesirable non-specific binding while the inclusion of the cRGD peptide targeting ligand increases the time of circulation and prevents additional degradation after binding to target cells. These in vivo properties help improve the signal-to-background ratio of imaged tissues, leading to higher resolution imaging.

Other features and advantages of the invention will be apparent from the following detailed description, and from the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE DRAWINGS

FIG. 1 a representation of the dye, targeting ligand, and conjugate imaging agent of the invention. FIG. 1 also shows two previously known near-infrared imaging dyes, ZW700-1-Forte (ZW700-1c) and ZW800-1.

FIG. 2 shows the normalized absorbance at 645 nm of ZW700-1-Forte in 37° C. buffered fetal bovine serum (FBS), pH 7.4 over time from 0 to 60 hours.

FIG. 3 shows the normalized absorbance at 768 nm of ZW800-1 in 37° C. buffered FBS, pH 7.4 over time from 0 to 60 hours.

FIG. 4 shows an overlay of the data from FIGS. 2 and 3.

FIG. 5 shows the full absorbance spectra for ZW700-1-Forte in 37° C. buffered FBS, pH 7.4 over time from 0 to 48 hours.

FIG. 6 shows the full absorbance spectra for ZW800-1 in 37° C. buffered FBS, pH 7.4 over time from 0 to 48 hours.

### DETAILED DESCRIPTION

The present disclosure relates, inter alia, to an imaging agent that is composed of a dye molecule optionally conjugated to a targeting ligand through a linking group. The imaging agent described herein is useful in, for example, the detection of abnormal or diseased biological tissues and cells. The conjugate is particularly useful for imaging whole organisms, because it has improved in vivo behavior, such as low non-specific binding to non-targeted tissues and ultrahigh stability, resulting in an improved signal-to-background ratio in connection with the detected optical signal. It is believed that these improved in vivo properties result from the balancing of formal charges on the conjugate, rendering a “charge-balanced” molecule having a net charge that is neutral or close to neutral.

### Definitions and Additional Embodiments

The following definitions will be useful in understanding the instant invention.

As used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used in the specification and claims, the singular form “a”, “an” and “the” include plural references unless the context clearly dictates otherwise.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or obvious from context, as used herein, the term “or” is understood to be inclusive.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

As used herein, the term “subject” or “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fish, parasites, microbes, and the like.

As used herein, the term “administration” or “administering” of the subject compound refers to providing a compound of the invention and/or prodrugs thereof to a subject in need of diagnosis or treatment.

As used herein, the term “carrier” refers to chemical compounds or agents that facilitate the incorporation of a compound described herein into cells or tissues.

As used herein, the term “acceptable” with respect to a formulation, composition or ingredient, as used herein, means having no persistent detrimental effect on the general health of the subject being treated.

As used herein, the term “diluent” refers to chemical compounds that are used to dilute a compound described herein prior to delivery. Diluents can also be used to stabilize compounds described herein.

The targeting ligand can be covalently attached to the reactive linking group of a dye compound of the invention through standard coupling procedures. For example, the carboxyl or activated carboxyl group of the reactive linking group can react with a nucleophilic functionality on the targeting ligand, such as an amine or alkoxy derivative, to form an amide or ester linkage. Additional details for the conjugation of dyes can be found in WO 2008/017074 and in Frangioni et al. Molecular Imaging, Vol. 1(4), 354-364 (2002), each of which is incorporated herein by reference in its entirety.

The cRGD targeting ligand can further include a molecular scaffold moiety to which the binding moiety and other groups can attach. For example, the molecule scaffold can bear one or more of the following: (1) a moiety designed to react with the reactive linking group of the dye to form a covalent bond, (2) a charge balancing moiety, such as any of the ionic groups described herein, and (3) a moiety that binds to the biological target. An example of a molecular scaffold is an adamantane derivative, such as described in U.S. Pat. App. Pub. No. 2006/0063834, which is incorporated herein by reference in its entirety, and illustrates the preparation of a targeting ligand that incorporates an adamantane scaffold. Specifically, the adamantane core holds (1) an amino group capable of reacting with the dye compounds, (2) a charge-balancing moiety that will neutralize a negative charge on the dye molecule, and (3) two moieties that bind to the biological target PSMA. For a description moieties that bind to PSMA, see, Humblet, V. et al. Mol. Imaging, 2005, 4:448-62; Misra P. et al. J. Nucl. Med. 2007, 48:1379-89; Chen, Y., et al. J. Med. Chem, 2008, 51:7933-43; Chandran, S. S., et al. Cancer Biol. Ther., 2008, 7:974-82; Banerjee, S. R., J. Med. Chem. 2008, 51:4504-17; Mease, R. C., et al. Clin. Cancer Res., 2008, 14:3036-43; Foss, C. A. et al. Clin. Cancer. Res., 2005, 11:4022-8, each of which is incorporated herein by reference in its entirety.

As used herein, the term “contacting” refers to the bringing together of substances in physical contact such that the substances can interact with each other. For example, when an imaging agent is “contacted” with tissue or cells, the tissue or cells can interact with the imaging agent, for example, allowing the possibility of binding interactions between the agent and molecular components of the tissue or cells. “Contacting” is meant to include the administration of a substance such as an imaging agent of the invention to an organism. Administration can be, for example, oral or parenteral.

As used herein, the term “ionic group” refers to a moiety comprising one or more charged substituents. The “charged substituent” is a functional group that is generally anionic or cationic when in substantially neutral aqueous conditions (e.g. a pH of about 6.5 to 8.0 or about physiological pH (7.4)). As recited above, examples of charged anionic substituents include anions of inorganic and organic acids such as sulfonate (—SO₃¹⁻), sulfinate, carboxylate, phosphinate, phosphonate, phosphate, and esters (such as alkyl esters) thereof. In some embodiments, the charged substituent is sulfonate. Examples of charged cationic substituents include quaternary amines (—NR₃⁺), where R is independently selected from C₁₋₆ alkyl, aryl, and arylalkyl. Other charged cationic substituents include protonated primary, secondary, and tertiary amines, and well as guanidinium. In some embodiments, the charged substituent is —N(CH₃)₃⁺.

As used herein, the phrase “non-ionic oligomeric or polymeric solubilizing groups” refers to soluble polymers such as, for example, polyethylene glycol, polypropylene glycol, polyethylene oxide and propylene oxide copolymer, a carbohydrate, a dextran, polyacrylamide, and the like. The solubilizing group can be attached by any desired mode. The point of attachment can be, e.g., a carbon-carbon bond, a carbon-oxygen bond, or a nitrogen-carbon bond. The attachment group can be, e.g., an ester group, a carbonate group, a ether group, a sulfide group, an amino group, an alkylene group, an amide group, a carbonyl group, or a phosphate group.

Some examples of solubilizing groups include polyethylene glycols, such as —(CH₂CH₂O)ₐ—H, —OC(═O)O(CH₂CH₂O)ₐH, —OC(═O)O(CH₂CH₂O)ₐCH₃, —O(CH₂CH₂O)ₐCH₃, and —S(CH₂CH₂O)₂CH₃, “a” being an integer between about 2 and about 25O. In some embodiments, “a” is 4 to 12 or 5 to 10. In further embodiments, “a” is 6, 7, or 8. Other examples of solubilizing groups include dextrans such as —OC(═O)O(dextran).

The solubilizing moiety can have an absolute molecular weight of from about 500 amu to about 100,000 amu, e.g., from about 1,000 amu to about 50,000 amu or from about 1,500 to about 25,000 amu.

Further examples of solubilizing groups include: —(CH₂)_{c}—(OCH₂CH₂)_{d}—OR^{a}, wherein “c” is 0 to 6, “d” is 1 to 200, and R^{a} is H or C₁₋₆ alkyl. In some embodiments, “c” is 1 to 4, “d” is 1 to 10, and R^{a} is H. In some embodiments, “d” is 6 or 7.

See WO 2008/017074, U.S. Ser. No. 12/376,243 (filed Feb. 3, 2009), and U.S. Ser. No. 12/376,225 (filed Feb. 3, 2009), each of which is incorporated herein by reference in its entirety, for a further description of suitable non-ionic oligomeric or polymeric solubilizing groups, and method for incorporating them into dyes.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Compounds of the invention can also include all isotopes of atoms occurring in the intermediates or final compounds. Isotopes include those atoms having the same atomic number but different mass numbers. For example, isotopes of hydrogen include tritium and deuterium.

The chemical substances represented herein by name, chemical formula, or structure are meant to include all stereoisomers, geometric isomers, tautomers, resonance structures, and isotopes of the same, unless otherwise specified.

The chemical substances described herein may be charged or include substituents with formal charges. When such chemical substances are represented as charged, it is understood that, unless otherwise specified, the charges are generally countered with an appropriate counterion. For example, chemical substances or functional groups having a charge of −I are understood to be countered with an ion have a +1 charge. Suitable counterions with +1 charge include Na+, K+, tetraalkylammonium ions, and the like. Conversely chemical substances or functional groups having a charge of +1 are understood to be countered with an ion having a-1 charge. Suitable counterions with −1 charge include F−, Cl−, Br−, I−, perchlorate, acetate, trifluoroacetate, and the like.

### cRGD-ZW700-1c

In one aspect, the invention provides a imaging agent comprising a ZW700-1c dye conjugated to a cyclic-RGD peptide targeting ligand. The conjugation can be done directly between the ZW700-1c dye and the cRGD targeting ligand or through a linking group.

In some embodiments, the imaging agent of the invention has the following structure.

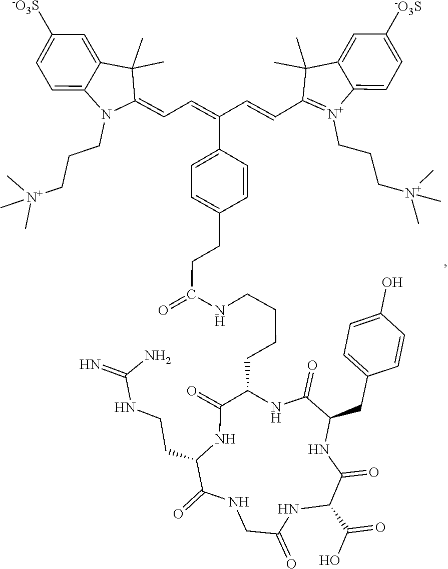

wherein L is an optional linking group.

The cRDG peptide targeting ligand can be covalently attached to a reactive group of the dye compound, or through an optional linking group (L), through standard coupling procedures. For example, the carboxyl or activated carboxyl group of the reactive linking group can react with a nucleophilic functionality on the targeting ligand, such as an amine or alkoxy derivative, to form an amide or ester linkage. Additional details for the conjugation of dyes can be found in WO 2008/017074 and in Frangioni et al. Molecular Imaging, Vol. 1(4), 354-364 (2002), each of which is incorporated herein by reference in its entirety. As used herein, “linking group” refers to any molecular entity having a molecular weight from about 50 to about 500 Da that is capable of conjugating with a targeting ligand (TL). In particular, the linking group includes at least one reactive group selected from a carboxylic acid group or anhydride or ester thereof, as well as an isothiocyanate group. In some embodiments, the linking group contains a carboxylic acid group. In some embodiments, the linking group is a PEG moiety or a straight or branched chain hydrocarbon moiety having between 2 and 12 carbon atoms. In some embodiments, the linking group includes a branching point, a reactive linking group, or other reactive group which can be used to further functionalize the imaging agent, for example, for inclusion of a radioisotope.

In particular embodiments, the imaging agent of the invention has the following structure.

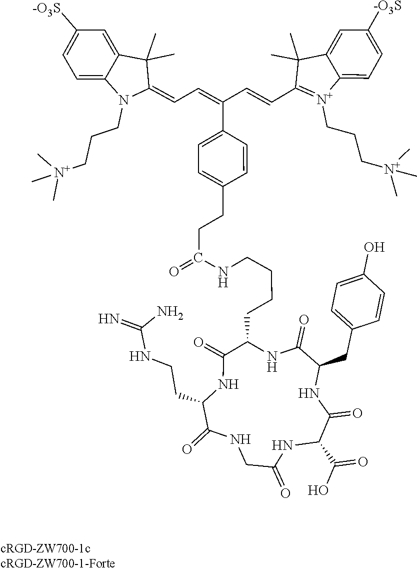

ZW700-1c can be prepared using the following scheme 1:

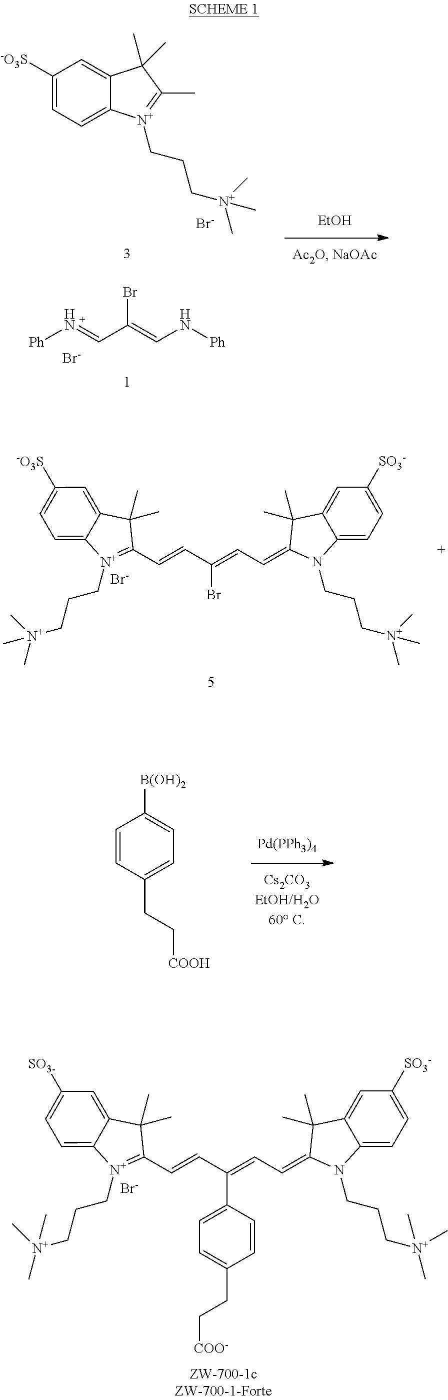

Additional means of synthesis can be found in Example 1 below. Still other means of synthesis can be found, for example, in Mol Imaging Biol (2016) 18:52-61 and in U.S. Pat. No. 10,201,621B2, each of which is incorporated herein by reference.

The present invention further provides methods for preparing conjugates suitable for the imaging methods described herein.

The present invention further provides methods of preparing a conjugate for imaging tissue or cells, wherein the conjugate includes a ZW700-1c and a cRGD targeting ligand. These methods include: (a) optionally modifying the ZW700-1c to include a linking group; (c) modifying the ZW700-1c and optionally the linking group to include one or more ionic groups to achieve a solubility of at least about 10 μM in 10 mM HEPES solution at pH 7.4; and (d) conjugating the cRGD targeting ligand to the ZW700-1c optionally through the linking group to form the conjugate, wherein the targeting ligand and the one or more ionic groups are selected so that the net charge of the conjugate is +1, 0, or −1, and wherein the signal-to-background ratio of fluorescent emission detected from the conjugate while imaging is at least about 1.1 Additional means of synthesis can be found in Example 1 below.

ZW700-1c, cRGD targeting ligands, and imaging agents can be isolated as salts, acids, bases, or combinations thereof. For example, dyes, conjugates, and imaging agents having multiple charged substituents can be isolated by introducing counterions and/or protons sufficient to counter the charges of the various substituents normally present in neutral pH so that the dye, conjugate, or imaging agent can be isolated, for example, as a solid substance.

The cRGD targeting ligand can be covalently attached to the reactive linking group of the dye compound of the invention through standard coupling procedures. For example, the carboxyl or activated carboxyl group of the reactive linking group can react with a nucleophilic functionality on the targeting ligand, such as an amine or alkoxy derivative, to form an amide or ester linkage. Additional details for the conjugation of dyes can be found in WO 2008/017074 and in Frangioni et al. Molecular Imaging, Vol. 1(4), 354-364 (2002), each of which is incorporated herein by reference in its entirety.

In certain embodiments, the imaging agent further comprises a PEG-moiety. Such moiety can be bound to the conjugate at any suitable structural location as would be understood by one of ordinary skill in the synthesis of such compounds. In addition, in certain embodiments, the PEG-moiety can be included as the optional linking group between ZW700-1c and the cRGD targeting ligand using methods known to one of ordinary skill in the art.

In certain embodiments, the imaging agent further comprises a radioisotope for either single-photon emission computed tomography (SPECT) or positron emission tomography (PET). Such radioisotope can be bound or further conjugated to the conjugate at any suitable structural location as would be understood by one of ordinary skill in the synthesis of such compounds.

In certain embodiments, the imaging agent further comprises a reactive linking group, such as NHS ester, sulfo-NHS ester, or a TFP ester. Such reactive linking groups can be bound or substituted onto the conjugated conjugate at any suitable structural location as would be understood by one of ordinary skill in the synthesis of such compounds.

### Imaging Methods

The methods of imaging tissue or cells include the following basic steps:
(a) contacting the tissue or cells with an imaging agent comprising a conjugate of a dye, the conjugate comprising a cRGD targeting ligand bound to the dye;
(b) irradiating the tissue or cells at a wavelength absorbed by the conjugate;
(c) detecting an optical signal from the irradiated tissue or cells,
wherein the signal-to-background ratio of the detected optical signal is at least about 1.1, thereby imaging the tissue or cells. In particular, the dye used in the invention is ZW700-1-c.

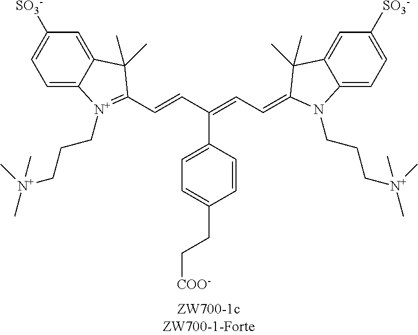

The imaging agent described herein is a substances to that can be used to image tissues or cells, such as those of a living organism, for purposes of diagnosis, therapy, image-guided surgery, and the like. In some embodiments, the organism is a mammal, such as a human.

The imaging agent of the invention contains a dye that is capable of absorption of electromagnetic radiation, typically in the ultraviolet (UV), visible, or near infrared (NIR) range. The imaging agent can also be capable fluorescent emission, such as in the visible or NIR range. The optical signal detected from the dye or conjugate can be, for example, absorption or fluorescent emission. In some embodiments, fluorescent emission from the dye is the primary optical signal detected for imaging purposes. In some embodiments, the dye has a peak absorbance at about 525 nm to about 850 nm, at about 550 nm to about 825 nm, about 600 nm to about 825 nm, about 700 nm to about 825 nm, or at about 750 nm to about 825 nm. In some embodiments, the dye has a peak fluorescent emission at about 700 nm to 875 nm, about 725 to about 850 nm, about 750 to about 850 nm, or about 775 to about 850 nm. In particular embodiments, the conjugated imaging agent of the invention has a peak fluorescent emission at about 700 nm.

The cRGD peptide targeting ligand is a targeting ligand which selectively binds to the biological target avi33 integrin.

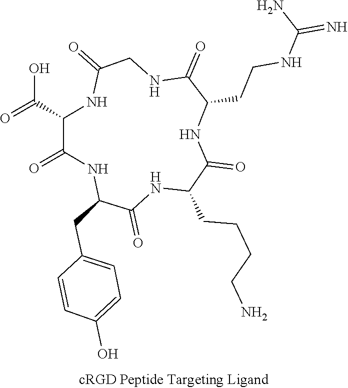 [SMILES: N=C(N)NCC[C@@H]1NC(=O)[C@H](CCCCN)NC(=O)[C@@H](Cc2ccc(O)cc2)NC(=O)[C@H](C(=O)O)NC(=O)CNC1=O]

It is known that this integrin is overexpressed by various tumors, and thus, these RGD targeting peptides enable the dyes to preferentially label tumors that overexpress these integrins. For example, the neovasculature of almost all solid tumors will upregulate certain integrins, and certain tumor cells also upregulate integrins on their surface.

In particular embodiments, the imaging agent of the invention has the following structure.

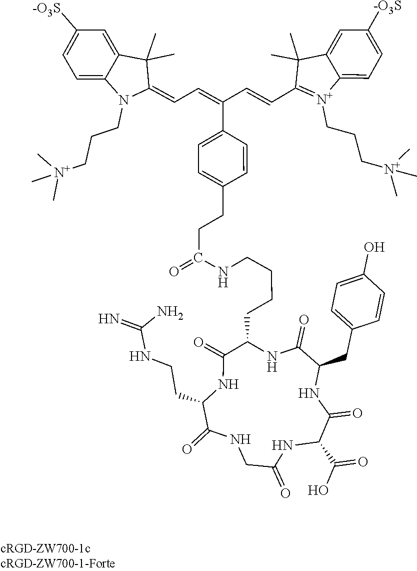

The imaging agent is generally “charge-balanced,” unless otherwise specified, which refers to having a net overall charge of zero, or close to zero, such as +1 or −1. Charge-balancing occurs when negatively charged substituents on the imaging agent are countered by the presence of an equal number, or close to an equal number, of positively charged substituents on the same molecule, and vice versa. In some embodiment, the net charge is 0 or +1. In some embodiments, the net charge is 0. In some embodiments, the net charge is +1. In further embodiments, the net charge is −1. The value “n” in the formulae provided herein represents net charge.

The imaging agent described herein generally has improved “signal-to-background ratio” (SBR) compared to presently known fluorescent imaging agents. The improvement in SBR is believed to be a result of improved in vivo properties due to “charge-balancing.” SBR is a measure of the intensity of the fluorescent signal obtained from a target (peak signal) over the measure of the intensity of the fluorescent signal obtained nearby the target (background signal), the target being the tissues or cells targeted by the imaging agent. SBR measurements can be readily obtained through routine measurement procedures. For fluorescent imaging systems, and other optical-type systems, digital images recording optical signals of the target facilitate SBR measurement. Higher SBR values are more desirable, resulting in greater resolution of the imaged tissues. In some embodiments, the imaging agents achieve an SBR of at least about 1.1 (i.e., peak signal is at least 10% over background). In further embodiments, the imaging agents achieve an SBR of at least about 1.2, at least about 1.3, at least about 1.4, at least about 1.5, at least about 1.6, at least about 1.7, at least about 1.8, at least about 1.9, or at least about 2.0. In yet further embodiments, the imaging agents achieve an SBR of about 1.1 to about 50, about 1.5 to about 30, about 2.0 to about 20, about 2.0 to about 5.0, or about 5.0 to about 10.

The imaging agent of the invention generally includes one or more ionic groups. In some embodiments, the imaging agents include two or more, three or more, four or more, or five or more ionic groups. Ionic groups serve to increase solubility of the generally hydrophobic dye portions of the imaging agent, thus, improving biodistribution. The ionic groups can be located on any portion of the imaging agent, such as the dye portion, the targeting ligand, or both.

The term “ionic group” refers to a moiety comprising one or more charged substituents. The “charged substituent” is a functional group that is generally anionic or cationic in substantially neutral aqueous conditions (e.g. a pH of about 6.5 to 8.0, or preferably about physiological pH (7.4)). Examples of charged anionic substituents include anions of inorganic and organic acids such as sulfonate (—SO₃¹⁻), sulfinate, carboxylate, phosphinate, phosphonate, phosphate, and esters (such as alkyl esters) thereof. In some embodiments, the charged substituent is sulfonate. Examples of charged cationic substituents include quaternary amines (—NR₃⁺), where R is independently selected from C₁₋₆ alkyl, aryl, and arylalkyl. Other charged cationic substituents include protonated primary, secondary, and tertiary amines, and well as guanidinium. In some embodiments, the charged substituent is —N(CH₃)₃⁺-. Further examples of ionic groups are described infra.

The imaging agent of the invention generally has good solubility in substantially neutral aqueous media, and in particular, blood and blood serum. In some embodiments, the imaging agent has a solubility in 10 mM HEPES solution, pH 7.4, of at least about 10 μM. In further embodiments, the imaging agent has a solubility in 10 mM HEPES solution, pH 7.4, of at least about 15 μM at least about 20 μM, at least about 25 μM, at least about 30 μM, at least about 40 μM, or at least about 50 μM.

The imaging agent of the invention generally can be neutral molecules or salts. For example, if the dye or dye conjugate is charged, the imaging agent can be or contain a salt or acid (or combination thereof) of the dye or dye conjugate. For positively charged dyes or conjugates, suitable counter ions include anions such as fluoride, chloride, bromide, iodide, acetate, perchlorate, PF₆⁻, and the like. For negatively charged dyes or conjugates, suitable counterions include cations such as Na⁺, K⁺, and quaternary amines.

The imaging agent of the invention has significant improvements with regard to stability over time, allowing for dramatically improved operability and use for imaging and mapping. Similarly, the stability of the imaging agent allows for increased accuracy during surgery as the signal does not degrade over time.

The imaging agent of the invention can be administered by any suitable technique, including both enteral and parenteral methods. In some embodiments, the imaging agents can be formulated into pharmaceutically acceptable formulations and administered intravenously to an organism for imaging. The dosed organism can be imaged using, for example, a FLARE™ Image-Guided Surgery System, which is a continuous-wave (CW) intraoperative imaging system that is capable of simultaneous, real-time acquisition and display of color video (i.e., surgical anatomy) and two channels of invisible NIR fluorescent (700 nm and 800 nm) light. The imaging system can irradiate the dosed organism with radiation absorbed by the imaging agent, and detect optical signals, such as NIR fluorescence, emanating from the targeted portions of the organism containing the imaging agent. The detected signals can be recorded and analyzed by obtaining digital images or video of the subject organism, thereby facilitating diagnostic procedures and image-guided medical techniques.

### Methods of Identifying and Imaging Tumors and Other Conditions Associated with Integrin Overexpression

The charge-balanced imaging agent of the is particularly advantageous because their behavior in vivo is believed to contribute to superior optical imaging properties. More specifically, the charge-balancing is believed to impart good biodistribution and clearance properties to the agents, and reduce undesirable non-specific binding. These in vivo properties help improve the signal-to-background ratio of imaged cells and tissues, leading to higher resolution imaging. Furthermore, the charge-balanced imaging agents of the inventions are cleared by the kidneys with great specificity as described in the examples.

Integrins are a family of cell adhesion receptors that provide for cell motility and invasion. The integrin family in humans comprise 18 α and β subunits which assemble into different functional heterodimers. Some integrins, act as mediators of angiogenesis in solid tumors. Integrin expression is important for tumor progression and metastasis by promoting tumor cell migration, invasion, proliferation, and survival. In addition to cancer, integrins are also highly expressed during many normal and abnormal processes, such as fibrosis, wound healing, and inflammation. Integrin αvβ3 expression has been established as a surrogate marker of angiogenic activity. Similarly, Integrin αvβ6 is a subtype of the integrin family that is expressed exclusively on epithelial cells. αvβ6 is usually expressed at low or undetectable levels in normal adult tissues but can be highly upregulated during pathological and physiological processes such as wound healing, fibrosis, inflammation, and cancer. RGD mimetics have been used in some studies as a targeting moiety to deliver integrin antagonists to a variety of cell types. However, continuous infusion of RGD peptides have been found to stimulate tumor growth (See, Reynolds, L. E. et al. Enhanced pathological angiogenesis in mice lacking β3 integrin or β3 and β5 integrins. Nature Med. 8, 27-34 (2002)). In addition, many RGD bound agents have expensive, complex, time-consuming and low-yield synthetic procedures. Similarly, with regard to imaging, degradation of agents accumulation of RGD bound agents in the bladder have been found to impair some imaging methods. Given their stability and clearance rates, the conjugates of the invention have the potential to provide a long-lasting visual identification and imaging of tumors which overexpress integrins while avoiding the setbacks of prolonged systemic administration of RGD peptide ligands.

As such, in a particular aspect, the charge-balanced imaging agent may be used to image or identify (for example, during surgery) integrin overproduction in the cells of a subject.

As such, in one aspect, the invention provides a method for imaging integrin producing cells in a subject, the method comprising:
(a) administering an imaging agent comprising a conjugate of a dye, the conjugate comprising a cRGD targeting ligand bound to the dye;
(b) irradiating cells suspected of overproducing integrin at a wavelength absorbed by the conjugate;
(c) and detecting an optical signal from the irradiated conjugate, thereby imaging the cells overproducing integrin in the subject.

In particular, the invention encompasses a method of identifying or imaging such cells wherein the signal-to-background ratio of the detected optical signal is at least about 1.1.

In particular embodiments, the cells are tumor cells. For example, and without limitation, the tumor cells are melanoma cells, sarcoma cells, musculoskeletal tumor cells, breast cancer tumor cells, renal cancer cells, rectal cancer tumor cells, bone metastases, neuroendocrine tumor cells, brain metastases, flioblastoma multiforme (GBM) cells, squamous cell carcinoma cells of the head and neck (SCCHN), or non-small cell lung cancer (NSCLC) cells. As such, the invention provides for a method of imaging such tumors in a subject.

In other embodiments, the cells are associated with inflammation or angiogenesis. For example, and without limitation, the cells may be associated with inflammatory lesions, endometriosis, ischemic injury, cardiovascular disease, neurovascular disease, myocardial infarction, moyamoya disease, stroke, atherosclerosis, or rheumatoid arthritis.

In one aspect, the invention provides a method for treating benign or malignant tumors in a subject, the method comprising:
(a) administering an imaging agent comprising a conjugate of a dye, the conjugate comprising a cRGD targeting ligand bound to the dye;
(b) irradiating suspected tumor cells at a wavelength absorbed by the conjugate;
(c) and detecting an optical signal from the irradiated conjugate, thereby imaging the tumor cells in the subject; and
(d) treating or removing the tumor cells from the subject.

In the methods of the invention, the imaging agent is administered at a predetermined dosage. The predetermined dosage amount is not particularly limited provided that the predetermined dosage is administered at least a minimum amount capable of being cleared by the kidneys within twelve hours. In particular embodiments, the predetermined dosage is 0.5 mg/kg of body weight; 0.25 mg/kg of body weight; 0.1 mg/kg of body weight; 0.05 mg/kg of body weight; 0.01 mg/kg of body weight; 0.005 mg/kg of body weight; or 0.001 mg/kg of body weight. In other embodiments, the predetermined dosage is 5.0 mg; 2.5 mg, 1.0 mg; 0.75 mg; 0.5 mg; 0.25 mg; or 0.1 mg.

The administration of the imaging agent of the invention can be by any means described herein and as generally acceptable to the patient and one of ordinary skill in the art. In particular, the administration of the charge-balanced imaging agent is intravenous.

The predetermined target amount is dependent on a variety of factors including, but not limited to the type of tumor or condition to be observed and the location of any cells desired to be imaged. As such, the predetermined amount is set by one of ordinary skill in the art prior to administration of the agent. Such factors include, but are not limited to, the determined dosage amount, the height, weight, age, body mass index, and gender of the patient or any combination thereof.

In particular embodiments, when the predetermined dosage amount is between about 2.5 mg and about 5.0 mg or greater, the predetermined target amount is 50% of the pre-determined dosage amount. In other embodiments, when the predetermined dosage amount is between about 0.5 mg and about 2.5 mg, the predetermined target amount is 60% of the pre-determined dosage amount. In still other embodiments, when the predetermined dosage amount is about 0.5 mg or less, the predetermined target amount is 80% of the pre-determined dosage amount.

### Applications, Properties, and Compositions

The conjugates described herein can be used for, e.g., planar optical, optical tomographic, endoscopic, photoacoustic, and sonofluorescent applications for the detection, imaging, and treatment of tumors and other abnormalities. The conjugates can also be used for localized therapy. This can be accomplished, e.g., by directing the conjugates to a desired target site, or allowing the conjugates to accumulate selectively in the target site; shining light of an appropriate wavelength to activate the agent. Thus, the new conjugates can be used to detect, image, and treat a section of tissue, e.g., a tumor.

In addition, the conjugates can be used to detect the presence of tumors and other abnormalities by monitoring the blood clearance profile of the conjugates, for laser assisted guided surgery for the detection of small micrometastases of, e.g., somatostatin subtype 2 (SST-2) positive tumors, and for diagnosis of atherosclerotic plaques and blood clots.

The conjugates can be formulated into diagnostic and therapeutic compositions for enteral or parenteral administration. Generally, these compositions contain an effective amount of the conjugate, along with conventional pharmaceutical carriers and excipients appropriate for the type of administration contemplated. For example, parenteral formulations include the conjugate in a sterile aqueous solution or suspension. Parenteral compositions can be injected directly into a subject at a desired site, or mixed with a large volume parenteral composition for systemic administration. Such solutions can also contain pharmaceutically acceptable buffers and, optionally, electrolytes, such as sodium chloride.

Formulations for enteral administration, in general, can contain liquids, which include an effective amount of the desired dye or dye conjugate in aqueous solution or suspension. Such enteral compositions can optionally include buffers, surfactants, and thixotropic agents. Compositions for oral administration can also contain flavoring agents, and other ingredients for enhancing their organoleptic qualities.

Generally, the diagnostic compositions are administered in doses effective to achieve the desired signal strength to enable detection. Such doses can vary, depending upon the organs or tissues to be imaged, and the imaging equipment being used. For example, Zeheer et al., Nature Biotechnology, 19, 1148-1154 (2001) uses 0.1 μmol/kg as a dose for IRDye78 conjugates in vivo. The diagnostic compositions can be administered to a patient systemically or locally to the organ or tissue to be imaged, and then the patient is subjected to the imaging procedure.

Generally, the conjugates or dye compounds absorb and emit light in the visible and infrared region of the electromagnetic spectrum, e.g., they can emit green, yellow, orange, red light, or near infrared light (“NIR”).

In some embodiments, the ZW700-1c dye emits and/or absorbs radiation having a wavelength from about 300 nm to about 1000 nm, e.g., from about 400 nm to about 900 nm, or from about 450 mu to about 850 nm. In particular embodiments, the ZW700-1c dye emits and/or absorbs radiation having a wavelength of about 700 nm

In some embodiments the conjugates and dye compounds have a maximum excitation and/or a maximum emission, measured in 10 mM HEPES solution, pH 7.4, of from about 525 mn to about 875 nm, e.g., from about 550 nm to about 825 nm, or from about 550 nm to about 800 nm.

The invention will be described in greater detail by way of specific examples. The following examples are offered for illustrative purposes, and are not intended to limit the invention or claims in any manner. A variety of noncritical parameters in these examples can be changed or modified to yield essentially the same results.

### EXAMPLES

### Example 1: Preparation of cRGD-ZW700-1c

### Preparation of ZW700-Br:

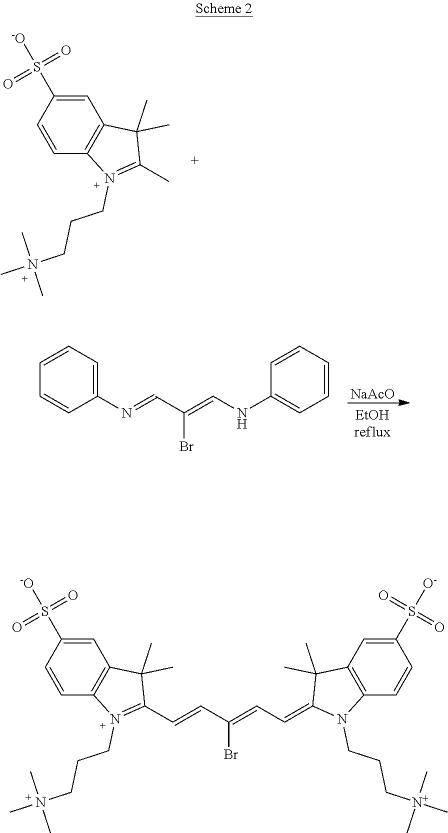

1. Prepare a 500 mL round bottom flask equipped with magnetic stirring, reflux condenser, and silicone oil bath (set to 95° C., monitored with Heidolph probe and maintained with Heidolph hot plate) and charge with 250 mL isopropanol, 18.77 g Quat sulfonate 3H indole potassium (In-house, 34.9 mmol), 5.00 g 700-Br (In-house, 16.6 mmol) and 4.25 g sodium acetate (Sigma S2889, 51.7 mmol). A dark suspension will be observed initially and then complete solution with heating.
2. After overnight heating, a precipitate will form (may be hard to observe in dark reaction solution).
3. Wash the reaction forward with isopropanol and filter the reaction through a Buchner funnel equipped with Whatman filter paper. Wash with isopropanol (80 mL washes at least 3 times) and then ethanol (1×50 mL) and then 1:1 Ethyl Acetate:Methanol (1×90 mL).
4. Transfer the powder to a tared bottle (record tare weight and whether cap is included) and dry overnight in a vacuum desiccator. 14.00 g (15.5 mmol, 93.4% yield based on TFA salt form) of a blue-indigo powder should obtained.

### Preparation of ZW700-1c:

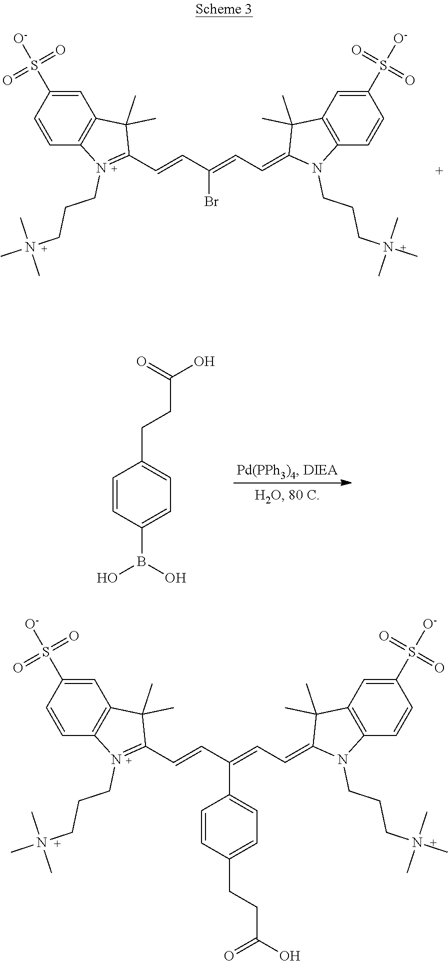

1. Prepare a 250 mL round bottom flask equipped with magnetic stirring, reflux condenser, and silicone oil bath (set to 80° C., monitored with Heidolph probe and maintained with Heidolph hot plate) and charge with 100 mL water, 1.50 g ZW700-Br (In-house, 1.72 mmol), 0.35 g 4-(2-carboxyethyl)benzene boronic acid (Alfa Aesar 10176898, 1.80 mmol), 0.15 g Tetrakis(triphenylphosphine) palladium (0) (Sigma SHBF5874V, 0.13 mmol) and 4.00 mL NMM (Sigma SHBF6578V, 36.3 mmol). A dark suspension will be observed initially and then complete solution with heating.
2. After reaction is confirmed complete by LC-MS, shut off heat and allow the reaction vessel to cool to room temperature. Filter the reaction and precipitate in 900 mL 1:1:1 Ethyl Acetate:Acetone:Ethanol 0.1% TFA.
3. Filter the precipitate through a Buchner funnel equipped with Whatman filter paper.
4. Transfer the powder to a tared bottle (record tare weight and whether cap is included) and dry overnight in a vacuum desiccator. 0.58 g (0.6 mmol, 34.8% yield based on TFA salt form) of a gold-green powder should be obtained.
5. If necessary recrystallization in water can be done at 30 mg/mL and allowing it to crystallize in refrigerator at 4 Celsius overnight.
   Preparation of cRGD-ZW700-1c
1. Prepare a round bottom flask equipped with magnetic stirring and charge with DMSO, 1.0 g ZW700-1C (1.16 mmol), cRGD-targeting ligand (3.65 mmol) and DIEA (Sigma, 1.72 mmol). A dark solution will be observed initially.
2. After 30 minutes, precipitate the crude reaction into 300 mL of 0.1% TFA in 1:1:1 Ethanol:Ethyl Acetate:Acetone and allow this to sit for a minimum of 20 minutes.
3. Filter the solids from the precipitation in step 2, wash with ethanol (2×25 mL) and place them in a vacuum desiccator overnight to dry. A powder should be obtained.

In certain embodiments, the imaging agent further comprises a PEG-moiety. Such moiety can be bound to the conjugate at any suitable structural location as would be understood by one of ordinary skill in the synthesis of such compounds. In addition, in certain embodiments, the PEG-moiety can be included as a linker between ZW700-1c and the cRGD targeting ligand using methods known to one of ordinary skill in the art. In such instances, the PEG moiety would be bound to either the ZW700-1C or the cRGD-targeting ligand prior to the preparation of the cRGD-ZW700-1c moiety. Such embodiments would be referred to as PEG-cRGD-ZW700-1c or cRGD-PEG-ZW700-1c depending on the location of the PEG Moiety

### Example 2: Imaging of Organisms

The FLARE™ Image-Guided Surgery System is a continuous-wave (CW) intraoperative imaging system that is capable of simultaneous, real-time acquisition and display of color video (i.e., surgical anatomy) and two channels of invisible NIR fluorescent (700 nm and 800 nm) light. Details of the theory, engineering, and operation of the imaging system has been described in detail previously. See, Tanaka, E., H. S. Choi, H. Fujii, M. G. Bawendi, and J. V. Frangioni, Image-guided oncologic surgery using invisible light completed: pre-clinical development for sentinel lymph node mapping. Ann Surg Oncol, 2006. 13:1671-81; De Grand, A.M. and J. V. Frangioni, An operational near-infrared fluorescence imaging system prototype for large animal surgery. Technol Cancer Res Treat, 2003. 2:553-562; and Nakayama, A., F. del Monte, R. J. Hajjar, and J. V. Frangioni, Functional near-infrared fluorescence imaging for cardiac surgery and targeted gene therapy. Molecular Imaging, 2002. 1:365-377, each of which is incorporated herein by reference.

Specifications for the FLARE™ Image-Guided Surgery System is provided in Table 1 below.

TABLE 1
FLARE ™ NIR Fluorescence Imaging System Specifications

| Category | Specification | Description |
|---|---|---|
| Physical | Size | Mobile Cart: 32″ W × 32″ D × 41.4″ H; Mast Height: |
| | | 82″ |
| | Weight | 675 lbs, including all electronics |
| | Arm | 6-degree-of-freedom; Reach: 43″-70″ from floor, |
| | | [symbol] ″ [symbol] |
| Electrical | Voltage and Plug | 120 V AC, 60 Hz; single NEMA 5-15 120 V/15 A AC |
| | | plug |
| | Current | 15 A max |
| | Grounding | Isolation transformer for all components; redundant |
| | | [symbol] |
| | Leakage Current | <300 μA (per AAMI/IEC #60601) |
| Sterility | Shield | Disposable acrylic shield with ≥95% transmission |
| | Drape | Disposable, custom-fit plastic drape bonded to shield |
| Light Source | Housing | Anodized aluminum with secondary 400 W cooling |
| | Elements | Custom 25 mm circular LED arrays w/ integrated linear |
| | Electronics | Custom passive and active boards with embedded |
| | | [symbol] |
| | Fluence Rates | 40,000 lx white light (400-650 nm), 4 mW/cm² of 700 |
| | | nm (656-678 nm) excitation light, 14 mW/cm² of 800 |
| Optics | Working Distance | 18″ from surface of patient |
| | Field-of-View | 2.2 W × 1.7 H cm to 15 W × 11.3 cm (adjustable zoom) |
| | Emission/Reflectance | Color Video (400-650 nm), 700 nm fluorescence |
| | Channels | (689-725 nm), 800 nm fluorescence (800-848 nm), all with |
| | Pixel Resolution | 640 × 480 for each camera |
| | System Resolution | 125 × 125 μm (x, y) to 625 × 625 μm (x, y) |
| | Display Refresh | Up to 15 Hz simultaneous acquisition on all 3 camera |
| | NIR Exposure Time | Adjustable from 100 μsec to 8 sec |
| Hands-Free | Optics | Automatic zoom/focus |
| | Control | 6-pedal footswitch |
| Monitors | Number | 2 cart-mounted 20″ for operator; 1 satellite 20″ on stand |
| | | for surgeon |

[symbol]

indicates data missing or illegible when filed

### Example 3: In Vivo Characterization of Dyes and Conjugates

For in vivo characterization, 40 pmol/g (average 10 nmol) of cRGD-ZW700-1c can be injected IV into 25 g athymic nude mice harboring xenograft human tumors. The FLARE™ imaging system can be set to a 665 nm excitation fluence rate of 1 mW/cm2. Simultaneous color video and NIR fluorescence (700 nm) images can be acquired pre-injection, every 1 sec for the first 20 sec then every 1 min for 2 h. Camera acquisition can be held constant (typically 100 msec) and chosen to ensure that all intensity measurements are within the linear range of the 12-bit Orca-AG (Hamamatsu) NIR camera. Blood can be sampled at 0, 1, 2, 5, 10, 15, 30, 60, and 120 min via tail vein. Intensity-time curves for all major organs and tissues can be quantified. The peak fluorescence intensity and time can be determined for each tumor/tissue/organ, along with the intensity in each at 1 h post-injection.

### Example 4: In Vitro Optical and Stability Properties of Dyes

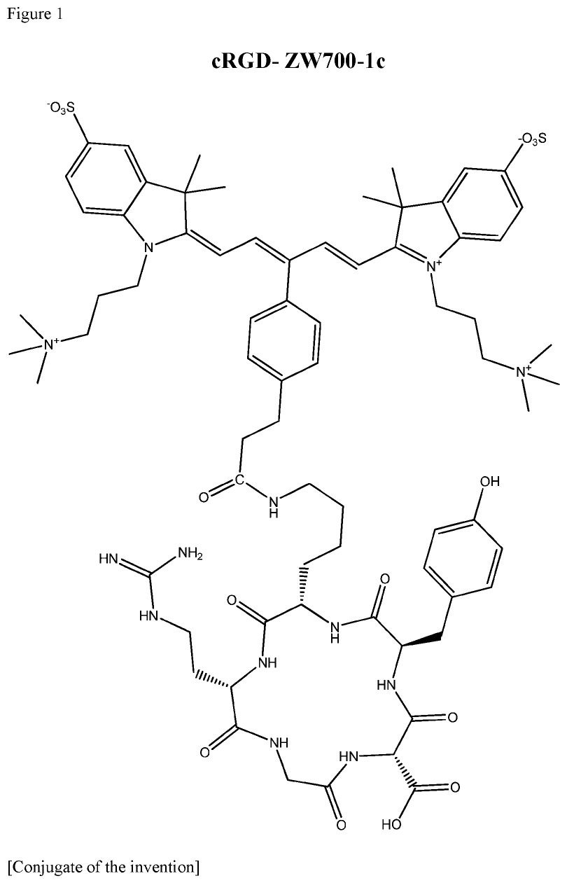

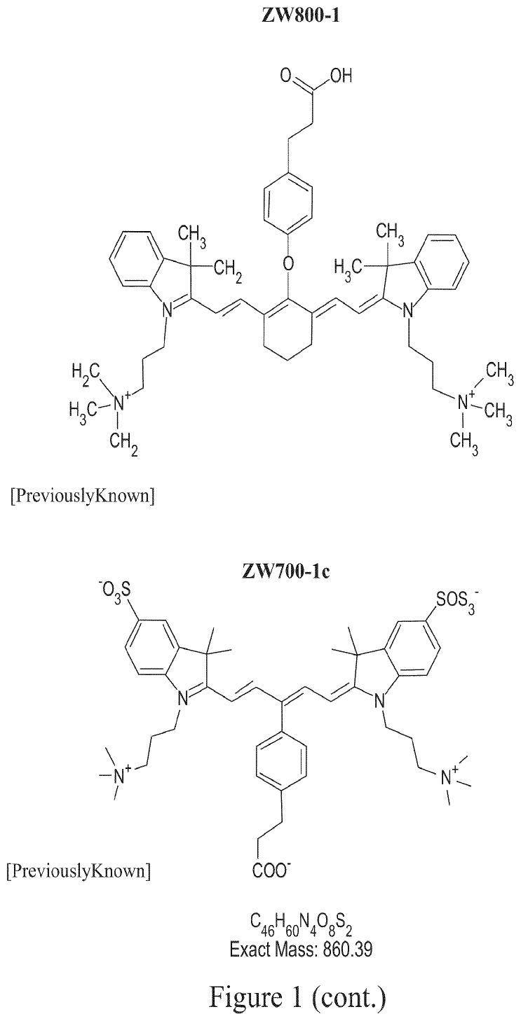

cRGD-ZW700-1c ZW700-1c, and ZW-800-1 are shown in FIG. 1 and are characterized with respect to their optical properties and stability in vitro. Commercial NIR fluorophores, such as IRDye™800-RS (RS-800), IRDye800-CW (CW-800), Cy5.5, and Cy7 have various degrees of sulfonation in order to achieve aqueous solubility.

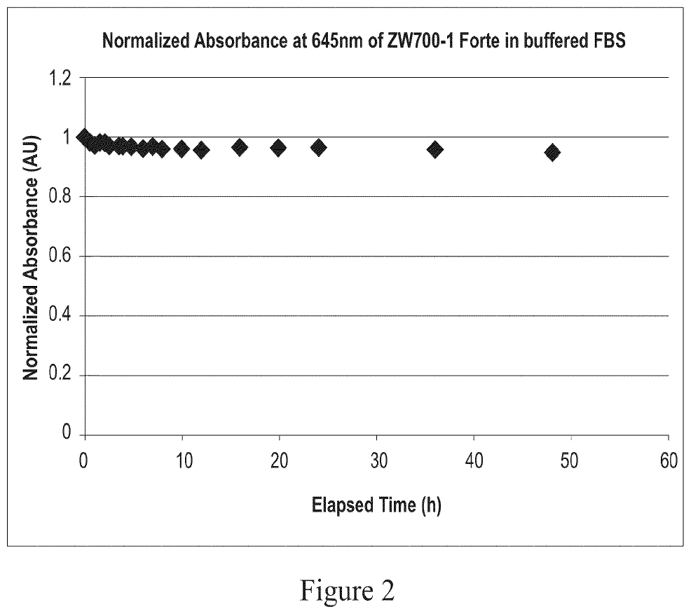

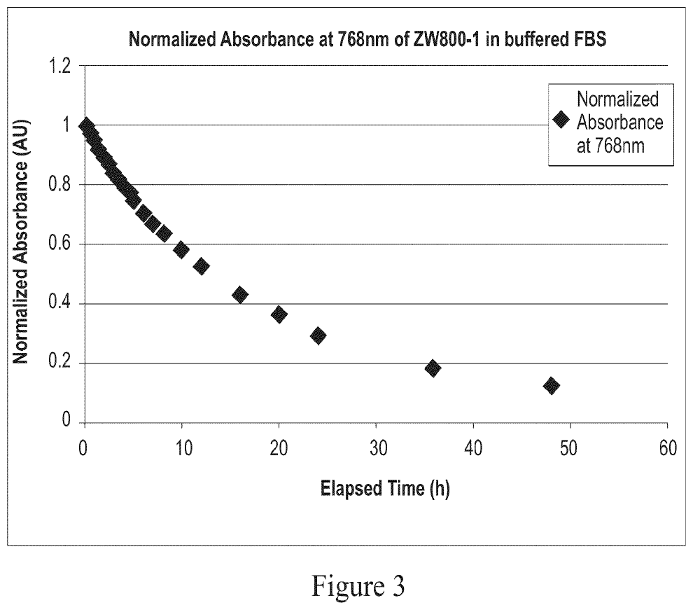

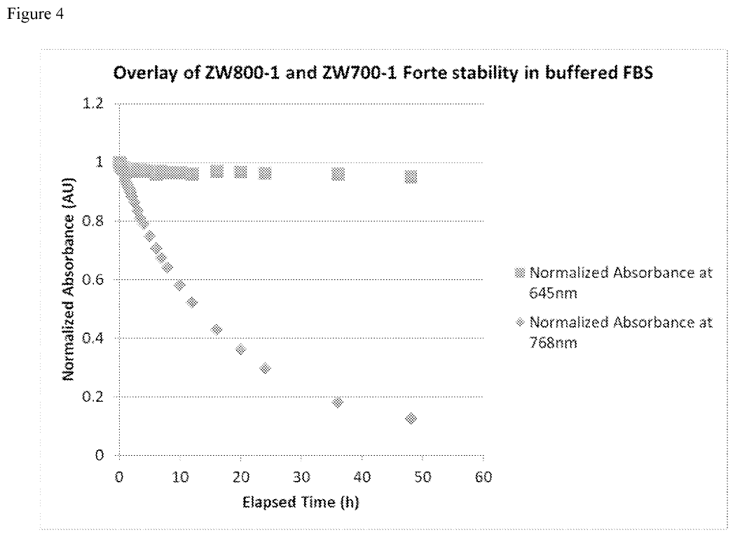

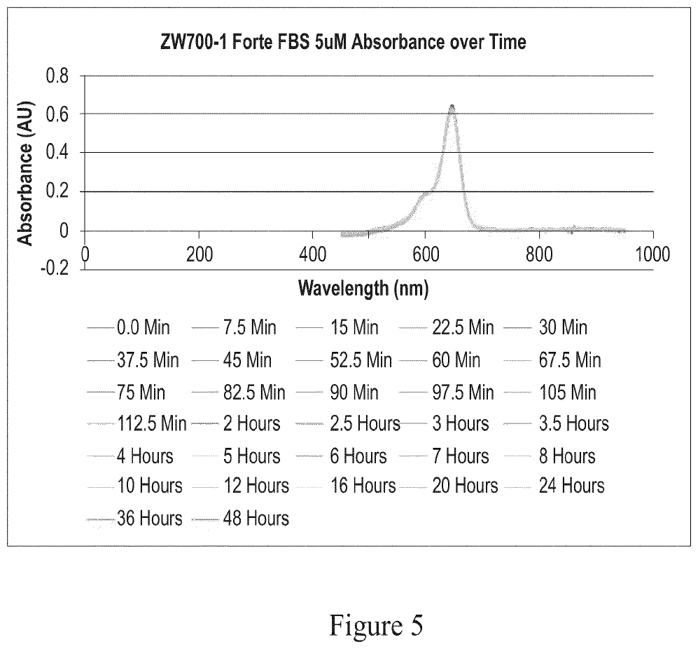

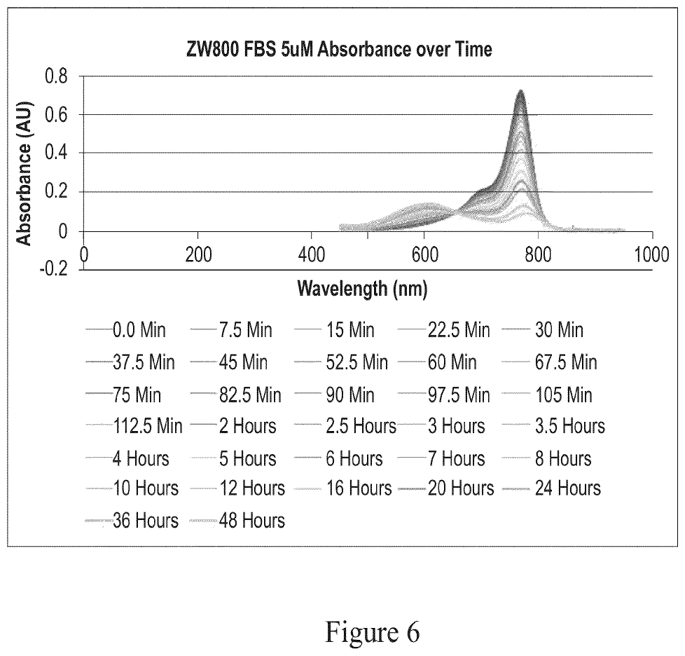

The normalized absorbance of ZW800-1 and ZW700-1c in 37° C. buffered FBS, pH 7.4 over time from 0 to 60 hours are shown in FIGS. 2-4. Note is made of the extreme stability of ZW700-1c compared to ZW800-1. Corresponding changes in absorption spectra during metabolism in warm serum is shown in FIGS. 5-6. Note is made of high stability of ZW700-1c over time, particularly as compared to ZW800-1.

cRGD is stable in serum for days. Data generated in the same way is expected to show that cRGD-ZW700-1c has further increased stability as compared to compared to ZW700-1c, ZW700-1c, ZW800-1 and cRGD-ZW800-1. In particular, cRGD-ZW700-forte is expected to have significant improvements in providing prolonged targeted imaging of tumors overexpressing integrins as compared to ZW700-1c, ZW800-1 and cRGD-ZW800-1. In addition, cRGD-ZW700-forte is expected to produce a brighter signal at a lower dose than as compared to ZW700-1c, ZW800-1 and cRGD-ZW800-1.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Those skilled in the art will recognize, or be able to ascertain using no more than routine experimentation, many equivalents to the specific embodiments of the invention described herein.

Such equivalents are intended to be encompassed by the following claims.

REFERENCES

| Year | PMID | Title | Authors | Citation |
|---|---|---|---|---|
| 2005 | 16085591 | Biodistribution and pharmacokinetics of | Beer et al. | J Nucl Med. |
| | | the alphavbeta3-selective tracer 18F- | | 2005 |
| | | galacto-RGD in cancer patients | | August; 46(8): |
| | | | | 1333-41 |
| 2008 | 18483090 | Phase I trial of the positron-emitting | Kenny et al. | J Nucl Med. |
| | | Arg-Gly-Asp (RGD) peptide radioligand | | 2008 |
| | | 18F-AH111585 in breast cancer patients | | June; 49(6): 879-86 |
| 2009 | 19330034 | Launching a novel preclinical | Paoloni | PLoS One. |
| | | infrastructure: comparative oncology | et al. | 2009; 4(3): e4972 |
| | | trials consortium directed therapeutic | | |
| | | targeting of TNFalpha to cancer | | |
| | | vasculature | | |
| 2009 | 19401596 | Imaging of integrin alpha(v)beta(3) | Schnell | Neuro Oncol. |
| | | expression in patients with malignant | et al. | 2009 |
| | | glioma by [18F] Galacto-RGD | | December; 11(6): |
| | | positron emission tomography | | 861-70 |
| 2012 | 22499613 | Biodistribution and radiation dosimetry | Doss et al. | J Nucl Med. |
| | | of the integrin marker 18F-RGD-K5 | | 2012 |
| | | determined from whole-body PET/CT | | May; 53(5): |
| | | in monkeys and humans | | 787-95 |
| 2013 | 23554506 | First experience of 18F-alfatide in lung | Wan et al. | J Nucl Med. |
| | | cancer patients using a new lyophilized | | 2013 |
| | | kit for rapid radiofluorination | | May; 54(5): 691-8 |
| 2014 | 25013808 | PET radiopharmaceuticals for imaging | Haubner et | Biomed Res Int. |
| | | integrin expression: tracers in clinical | al. | 2014; 2014: 871 |
| | | studies and recent developments | | 6 09 |
| 2015 | 26199649 | Pilot Prospective Evaluation of | Mi et al. | Theranostics. |
| | | (18)F- Alfatide II for Detection of | | 2015 Jul. |
| | | Skeletal Metastases | | 12; 5(10): |
| | | | | 1115-21. |
| 2015 | 26062933 | Biodistribution of the ¹⁸F-FPPRGD₂ PET | Minamimot | Eur J Nucl Med |
| | | radiopharmaceutical in cancer patients: | o et al. | Mol Imaging. |
| | | an atlas of SUV measurements | | 2015 |
| | | | | November; 42(12): |
| | | | | 1850-8 |
| 2016 | 26722375 | Clinical Application of Radiolabeled RGD | Chen et al. | Theranostics. |
| | | Peptides for PET Imaging of Integrin | | 2016 Jan. |
| | | ανβ3 | | 1; 6(1): 78-92 |
| 2016 | 26922351 | RGD-K5 PET/CT in patients with | Chen et al. | Eur J Nucl Med |
| | | advanced head and neck cancer treated | | Mol Imaging. |
| | | with concurrent chemoradiotherapy: | | 2016 |
| | | Results from a pilot study | | August; 43(9): |
| | | | | 1621-9 |
| 2016 | 27164900 | [(68)Ga]NODAGA-RGD - Metabolic | Haubner et | Eur J Nucl Med |
| | | stability, biodistribution, and dosimetry | al. | Mol Imaging. |
| | | data from patients with hepatocellular | | 2016 |
| | | carcinoma and liver cirrhosis | | October; 43(11): |
| | | | | 2005-13 |
| 2016 | 26611425 | Pilot prospective evaluation of | Minamimotoet | Eur J Nucl Med |
| | | (18)F- FPPRGD2 PET/CT in patients | al. | Mol Imaging. |
| | | with cervical and ovarian cancer | | 2016 |
| | | | | June; 43(6): |
| | | | | 1047-55 |
| 2017 | 28523582 | First in-human radiation dosimetry | Gnesin et | EJNMMI Res. |
| | | of (68)Ga-NODAGA-RGDyK | al. | 2017 |
| | | | | December; 7(1): 43. doi |
| 2019 | 30187104 | Pretreatment PET/CT imaging of | Li et al. | Eur J Nucl Med |
| | | angiogenesis based on (18)F-RGD tracer | | Mol Imaging. |
| | | uptake may predict antiangiogenic | | 2019 |
| | | response | | April; 46(4): |
| | | | | 940-947 |
| 2019 | 30392070 | [(99m)Tc]3PRGD(2) for integrin | Zheng et al. | Ann Nucl Med. |
| | | receptor imaging of esophageal cancer: | | 2019 |
| | | a comparative study with [(18)F]FDG | | February; 33(2): |
| | | PET/CT | | 135-143 |
| 2019 | 30254189 | A Phase II Trial of Pazopanib in | Kim et al. | Oncologist. 2019 |
| | | Patients with Metastatic Alveolar Soft | | January; 24(1): 20-e29 |
| | | Part Sarcoma | | |
| 2020 | 31519800 | A Clinical Feasibility Study to Image | Lobeek et | J Nucl Med. |
| | | Angiogenesis in Patients with | al. | 2020 |
| | | Arteriovenous Malformations | | February; 61(2): |
| | | Using (68)Ga-RGD PET/CT | | 270-275 |
| 2022 | 35657429 | A prospective head-to-head comparison | Jiang et al. | Eur J Nucl Med |
| | | of 68Ga-NOTA-3P-TATE-RGD and 68Ga- | | Mol Imaging. |
| | | DOTATATE in patients with | | 2022 |
| | | gastroenteropancreatic neuroendocrine | | October; 49(12): |
| | | tumours | | 4218-4227 |

The contents of all patent, patent applications, and publications cited herein are incorporated herein by reference in their entireties.

What is claimed is:

1. An imaging agent dye comprising a ZW700-1c conjugated to a cyclic-RGD peptide targeting ligand.

2. An imaging agent comprising a conjugate having the formula: 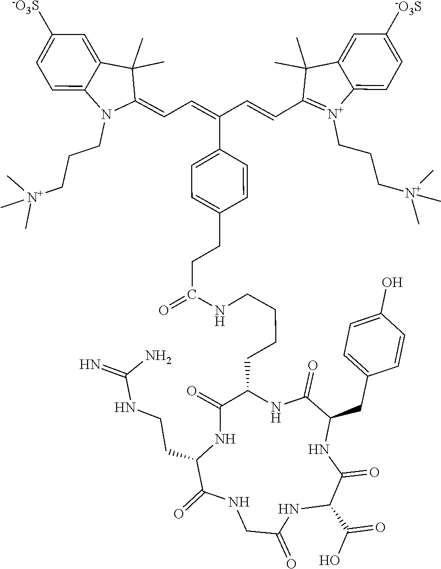
or a salt, solvate, or hydrate thereof.

3. A method of imaging cells, the method comprising:
(a) contacting cells with an imaging agent, wherein the imaging agent is: 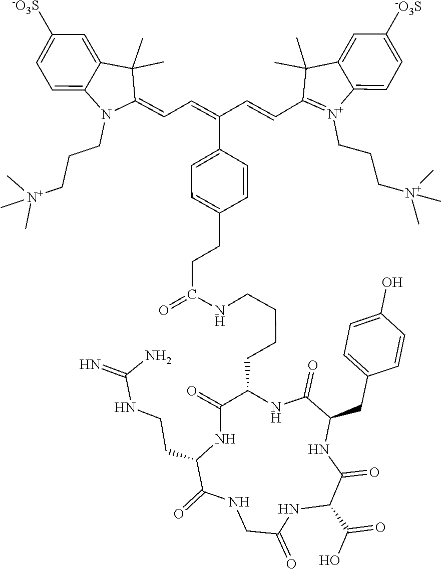
wherein L is an optional linking group;
(b) irradiating the cells at a wavelength absorbed by the imaging agent;
(c) and detecting a signal from the compound, thereby imaging the cells.

4. The method of claim 3, wherein the cells are or are suspected of overproducing one or more integrins.

5. The method of claim 4, wherein the cells are or are suspected of overproducing integrin αvβ3 or integrin αvβ6.

6. The method of claim 4, wherein the cells are tumor cells, inflammatory cells, or cells undergoing angiogenesis.

7. The method of claim 3, wherein the imaging agent is administered to an organism comprising or suspected of comprising the cells.

8. The method of claim 7, wherein the organism is human.

9. The method of claim 3, wherein the imaging agent has peak absorbance at about 600 nm to 850 nm.

10. The method of claim 3, wherein the tissue or cells is imaged in vivo.

11. The method of claim 3, wherein the imaging agent further comprises a PEG-moiety.

12. The method of claim 11, wherein the linking group is the PEG-moiety.

13. The method of claim 3, wherein the imaging agent further comprises a radioisotope for either single-photon emission computed tomography (SPECT) or positron emission tomography (PET).

14. The method of claim 3, wherein the imaging agent comprises a reactive linking group, such as NHS ester, sulfo-NHS ester, or a TFP ester.
